(19) United States
(12) Patent Application Publication
(10) Pub. No.: US 2022/0366370 A1
(43) Pub. Date: Nov. 17, 2022

(54) HYPERGRAPHIC SELF-DEFINING COMMUNICATIONS GROUPS OF AN ISOMORPHIC STRUCTURE

(71) Applicant: Mutualink, Inc., Wallingford, CT (US)

(72) Inventor: Joseph R. MAZZARELLA, Tolland, CT (US)

(73) Assignee: Mutualink, Inc., Wallingford, CT (US)

(21) Appl. No.: 17/318,714

(22) Filed: May 12, 2021

(51) Int. Cl. G06Q 10/10; H04M 3/51; H04L 29/08; G06F 40/174; G06F 3/0482; G06F 3/0484; G06Q 50/00

(52) U.S. Cl. CPC G06Q 10/103; H04M 3/5116; H04L 67/141; G06F 40/174; G06F 3/0482; G06F 3/0484; G06Q 50/01; G06Q 10/105; G06Q 50/26

(57) ABSTRACT

Some embodiments include an electronic device of a first agency configured to establish communications with a second agency via an interoperability communications network. The electronic device can include a processor configured to create an isomorphic structure including a first and a second node element in a hierarchy. The processor can instantiate an interactive visual chart (IVC) for the isomorphic structure that is activated, where the first and second node elements correspond to a first and second communications group identifiers (IDs). The processor can transmit a first invitation including the instantiated IVC for the activated isomorphic structure, and in response to the first invitation being accepted, instantiate a first communications group corresponding to the first communications group ID and a second communications group corresponding to the second communications group ID, where the first member and the second member are peers corresponding to the first node element of the activated isomorphic structure.

### BACKGROUND

### Field

The embodiments generally relate to self-defining communications groups, and in particular, hypergraphic self-defining communications groups utilizing isomorphic structures.

### Background

Modern principles of emergency management require a coordinated multiagency, multiple discipline response framework. These principles are captured within the US Department of Homeland Security's National Response Framework (NRF) and its corollary policies and guidelines, such as the National Incident Management System (NIMS), National Emergency Communications Plan, (NECP) and National Infrastructure Protections Plan (NIPP). While the principles are defined, implementing them in response to an emergency can be difficult.

### BRIEF SUMMARY OF THE INVENTION

An incident communications network enables interoperable communications among communications resources controlled by multiple organizations or individuals during an incident involving emergency or pre-planned multi-organization communications in which a communications resource is controlled by an administrator within an organization, or by an individual. The incident communications network includes interoperability workstation (IWS) controllers that control communications resources and enable a user a means to control and interface with the incident communications network.

U.S. Pat. No. 7,643,445, entitled Interoperable Communications System and Method of Use, issued on Jan. 5, 2010, and U.S. Pat. No. 8,320,874, entitled System and Method for Establishing an Incident Communications Network, issued on Nov. 27, 2012, both of which are incorporated by reference in their entirety, describe systems and methods for providing an interoperable communications system (“interop system,” also referred to as an Incident Communications Network) including a plurality of otherwise disjunct or disparate communications systems that addressed the deficiencies of prior art systems. The '445 and '874 patents specifically describe methods for establishing an incident communications network that enables interoperable communications among communications resources controlled by multiple organizations during an incident involving emergency or pre-planned multi-organization communications where a communications resource is controlled by an administrator within an organization.

Additionally, U.S. Pat. No. 8,364,153, entitled Mobile Interoperability Workstation Controller Having Video Capabilities within an Incident Communications Network, issued on Jan. 29, 2013, (“Mobile IWC Patent”) which is also incorporated herein by reference in its entirety, extends the concepts of the '445 and '874 patents. Namely, the Mobile IWC Patent includes enhanced video capture and streaming capabilities that are integrated with incident information and events to facilitate improved management and analysis of incidents or events in which an incident communications network is employed.

U.S. Pat. No. 8,811,940, entitled Dynamic Asset Marshalling Within an Incident Communications Network, issued on Aug. 19, 2014, (“Marshalling Patent”) which is also incorporated herein by reference in its entirety, extends the concepts of the '445 and '874 patents. Namely, the Marshalling Patent provides systems and methods that marshal resources into an incident communications network based on a variety of factors, such as the type of incident and the type of resource being marshaled.

U.S. Pat. No. 8,929,851, entitled System and Method for Establishing an Incident Communications Network (“'851 patent”), issued on Jan. 6, 2015, which is incorporated herein by reference in its entirety, extends the concepts of the '445 and '874 patents, Marshalling Patent, and the Mobile IWC Patent.

The incident communications network is described in the '445, '874, and '851 patents, Marshalling Patent, and the Mobile IWC Patent that are incorporated herein by reference.

Some embodiments include a system, method, and computer program product for instantiating hypergraphic self-defining communications groups according to an isomorphic structure activated during an incident. For example, an agency or organization can create an isomorphic structure such as an Incident Command System (ICS) structure directed to chemical and/or biological catastrophes. In the event of an incident (e.g., a mass casualty chemical and/or biological catastrophic event) that is pertinent to the isomorphic structure, some embodiments enable a first agency IWS to instantiate an instance of the isomorphic structure and create two or more communications groups (e.g., SIP protocol sessions) that correspond to node elements of the instantiated instance of the isomorphic structure. The first agency can invite a second agency to instantiate a corresponding isomorphic structure (e.g., a mass casualty chemical and/or biological catastrophic event) of the second agency, that enables members of the second agency to communicate with their respective peers in the first agency via the respective communications groups created.

Some embodiments include a system for a first agency, such as an IWS that includes a transceiver and a processor coupled to the transceiver. The processor establishes, via the transceiver, communications with a second agency via an interoperability communications network. The processor creates an isomorphic structure comprising a first and a second node element in a hierarchy, and instantiates a temporal instance of the isomorphic structure. The processor an interactive visual chart (IVC) for the instantiated temporal instance of the isomorphic structure, where the first node element corresponds to a first communications group identifier (ID) and the second node element corresponds to a second communications group ID. The processor transmits, via the transceiver, a first invitation including the instantiated IVC for the activated temporal instance of the isomorphic structure, and in response to the first invitation being accepted, instantiate a first communications group corresponding to the first communications group ID and a second communications group corresponding to the second communications group ID. The first communications group includes a first member of the first agency and a second member of the second agency, where the first member and the second member are peers corresponding to the first node element of the activated temporal instance of the isomorphic structure. The activated isomorphic structure can be an Incident Command System (ICS) structure.

To populate the IVC for the activated temporal instantiation of the isomorphic structure with members of the first agency, the processor transmits, via the transceiver, a second invitation to a first device of the first member of the first agency to accept a function corresponding to the first node element. In response to the second invitation being accepted, the processor assigns the first communications group ID corresponding to the first node element to the first device or the first member. The assignment can change for example, with a change in a work shift. The processor transmits, via the transceiver, a third invitation to the first device of the first member to continue to accept the function corresponding to the first node element (e.g., to continue working in the same position for another work shift or not.) In response to the third invitation being denied (e.g., the first member is changing the assignment with a work shift), the processor transmits a fourth invitation to another device of another member of the first agency to accept the function corresponding to the first node element. In response to the fourth invitation being accepted (e.g., the other member replaces the first member), the processor reassigns the first communications group ID corresponding to the first node element to the other device or the other member.

Some embodiments include a graphical user interface (GUI) coupled to the processor, where the GUI comprises hypergraphic selectable items corresponding one or more created isomorphic structures including the isomorphic structure. The processor receives, via the GUI, a first selection of a first hypergraphic selectable item corresponding to the isomorphic structure and activates a temporal instantiation of the isomorphic structure. The processor receives, via the GUI, a selection of a second hypergraphic selectable item corresponding to the first node element of the instantiated IVC for the activated temporal instantiation of the isomorphic structure. In response to the reception of the selection of the second hypergraphic selectable item, the processor transmits, via the transceiver, a second invitation to a first device of the first member of the first agency to accept a function corresponding to the first node element. In response to the second invitation being accepted, the processor assigns the first communications group ID to the first device or the first member. The first communications group ID can correspond to: a push to talk radio network, push to talk over mobile network, a voice or video conferencing call network.

In some embodiments the processor instantiates parallel sessions that include peer members from the first agency and a third agency coupled to the interoperability communications network, where the peer members correspond to respective node elements in the hierarchy of the activated temporal instance of the isomorphic structure.

In some embodiments the processor creates a member directory that includes a first agency subdirectory, publishes the first agency subdirectory, receives a second agency subdirectory, and updates the member directory to include the second agency subdirectory. In some embodiments the processor publishes one or more incident types supported by the first agency, receives incident types supported by the second agency, and updates the member directory to include the incident types supported by the second agency.

Further embodiments, features, and advantages, as well as the structure and operation of the various embodiments, are described in detail below with reference to accompanying drawings. It is noted that the embodiments are presented herein for illustrative purpose only. Additional embodiments will be apparent to persons skilled in the relevant art(s) based on the teachings contained herein.

### BRIEF DESCRIPTION OF THE DRAWINGS/FIGURES

The accompanying drawings, which are included to provide a further understanding of the invention and are incorporated in and constitute a part of this specification, illustrate embodiments of the invention and together with the description serve to explain the principles of the invention.

FIG. 1A illustrates an example system for establishing an interoperability communications group during an incident, in accordance with some embodiments of the disclosure.

FIG. 1B illustrates an example system for hypergraphic self-defining communications groups of an isomorphic structure during an incident, in accordance with some embodiments of the disclosure.

FIG. 2 illustrates an example system instantiating pre-defined communications groups during an incident, in accordance with some embodiments of the disclosure.

FIG. 3 illustrates an example system instantiating hypergraphic self-defining communications groups of an isomorphic structure during an incident, in accordance with some embodiments of the disclosure.

FIG. 4 illustrates an example method for instantiating hypergraphic self-defining communications groups of an isomorphic structure during an incident, in accordance with some embodiments of the disclosure.

FIG. 5 illustrates an example method for populating a hypergraphic interactive visual chart (IVC) of an isomorphic structure during an incident, in accordance with some embodiments of the disclosure.

FIG. 6 illustrates an example computer system for implementing some embodiments or portion(s) thereof.

FIG. 7 illustrates an example Incident Command System (ICS) structure for hospital use, according to some embodiments or portion(s) thereof.

FIG. 8 illustrates an example block diagram of a system for establishing hypergraphic self-defining communications groups of an isomorphic structure during an incident, in accordance with some embodiments of the disclosure.

### DETAILED DESCRIPTION

Some embodiments include a system, method, and a computer program product that instantiates hypergraphic self-defining communications groups of an isomorphic structure during an incident, where the communications groups enable communications among peers in different agencies, where the communications groups correspond to node elements of an activated temporal instantiation of the isomorphic structure.

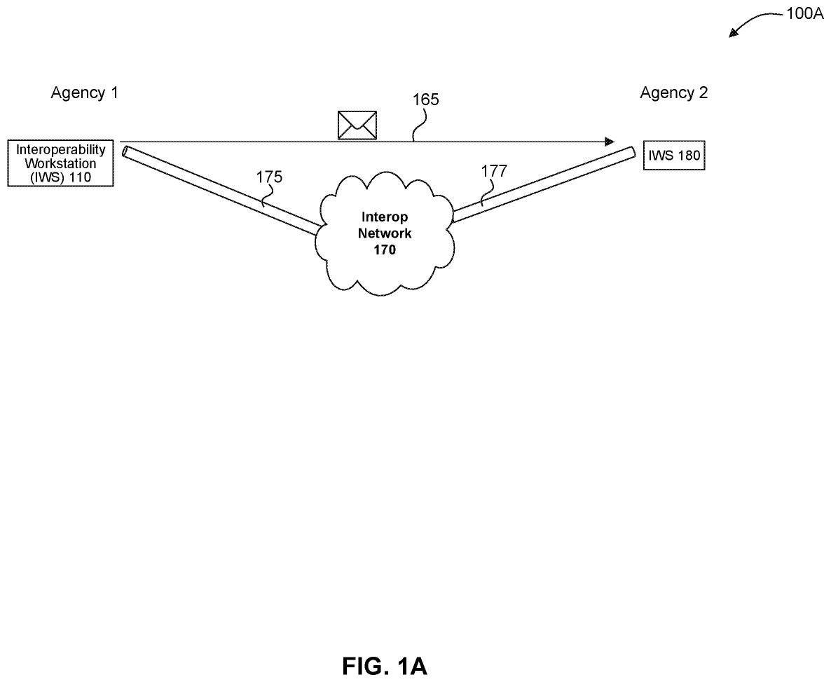

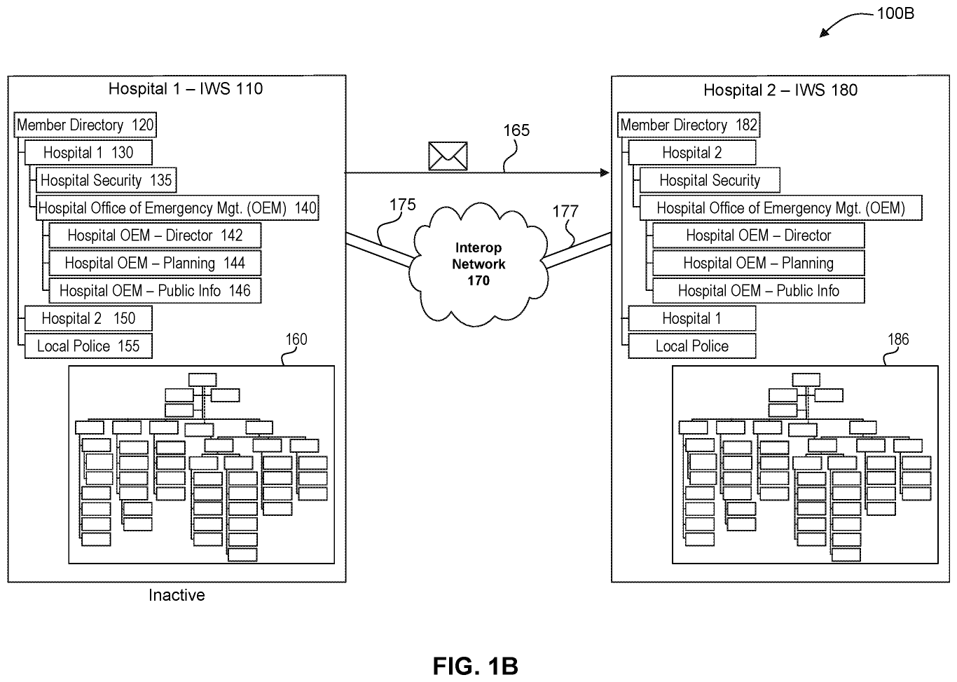

FIG. 1A illustrates example system 100A for establishing an interoperability communications group during an incident, in accordance with some embodiments of the disclosure. System 100A includes interoperability workstation (IWS) 110 of Agency 1 and IWS 180 of Agency 2. In the event of an incident (e.g., an explosion), IWS 110 can transmit invitation message 165 to IWS 180 of Agency 2 to invite Agency 2 to include one or more communication resources of Agency 2 to join a communications group. The one or more communication resources (e.g., land mobile radio devices, smart phone devices not shown) are communicatively coupled to IWS 180. If IWS 180 accepts invitation message 165 (e.g., by transmitting an acceptance message to IWS 110 (not shown.)) then a communications group (e.g., a session based on SIP protocol) is established between Agency 1 and Agency 2 via connections 175 and 177 with interop network 170 (e.g., an IP network.) In an example, Agency 1 and Agency 2 can be Hospital 1 and Hospital 2.

The expansive framework of emergency management includes an Incident Command System (ICS) promulgated by the US Department of Homeland Security-Federal Emergency Management Agency (FEMA). The ICS sets out a hierarchal organizational command structure constructed from rules premised on functional domains under which a function, a purpose or other distinctions define nodes, sub-nodes, nodes of sub-nodes, ad infinitum. To adopt an ICS, the capabilities and functions of an organization are conformed to a generalized ICS node structure, its hierarchical relations and defined functional divisions, and where not capable of being conformed, an organization may modify its ICS node structure and organizational relationships to best conform to the organization's functional divisions and capabilities.

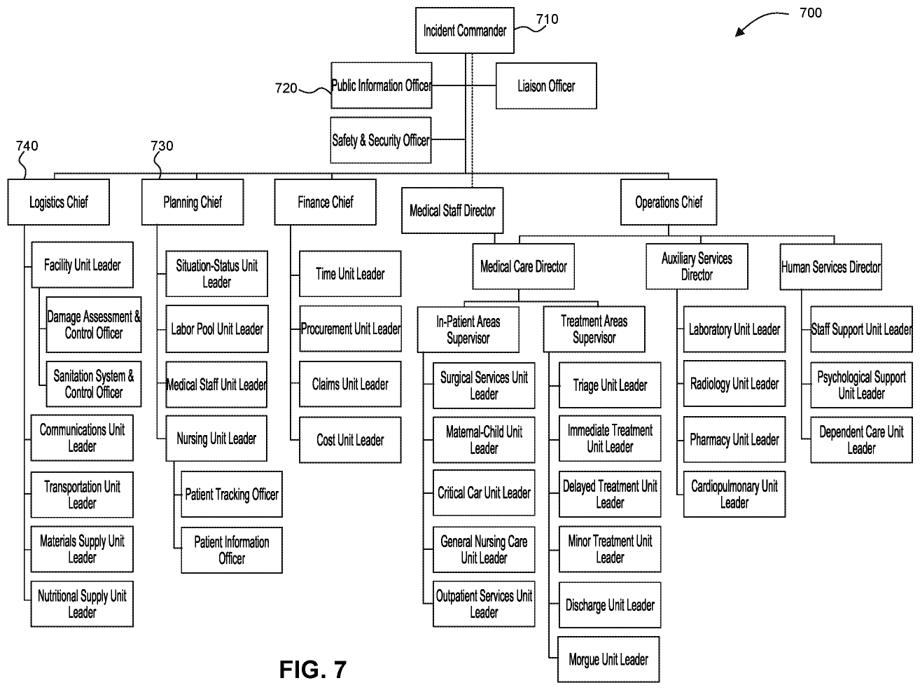

FIG. 7 illustrates example 700 of an ICS structure for hospital use, according to some embodiments or portion(s) thereof. The ICS structure of example 700 is an isomorphic structure. Example 700 is a node that includes one or more node elements. Example 700 includes node element, Incident Commander 710, at the highest level, and under the command level there are defined functional node elements that can be sections such as Public Information Officer 720, Logistics Chief 740, and Planning Chief 730. Within each node element there are defined positions such as directors, leaders, chief and other positional designations descriptive of job function or purpose. The connection of node elements to other node elements in a hierarchical relation or grouping is further defined by general span of control rules that establish the number of preferred subordinate relations that may exist from one node element to its sub-nodes in a dynamic cascading structure. Some sections include various subsections or branches, and some branches include various sub-branches or units. The relational structures may be further divided and made distinct. Some can be grouped into organizational units known as groups, task forces, and specialists.

Morphologically, the ICS structure and rules of construction may be translationally conceptualized to be a form of distinct vertices (e.g., ICS nodes) and connected edges (e.g., ICS node hierarchical relations) that generate isomorphic structures. The parent-child relations among ICS nodes are created by a hierarchical command rule under which one ICS node is subordinate to its senior. Each ICS node is comprised of at least one node element that is defined as the command or senior position (e.g., the Control Element) and all other positional node elements being members of the ICS node are directly or subordinate to the Control Element. Also, there may be other subordinate node elements which have control functions that control subsets of positional node elements, thus have their own Control Element.

As discussed, supra, the ICS rules of construction generate distinct isomorphic instantiations of ICS(s) structures (e.g., example 700) when created by distinct organizations or groupings of organizations. These instantiations are sufficiently similar among other distinct organizations or groups of organizations to enable the logical mapping of general functions or special functions (e.g., node elements including but not limited to incident commander 710, public information officer 720, planning chief 730, logistics chief 740) from one distinct organization to another for coordinated or joint actions.

An ICS structure and its various ICS nodes and node elements can be developed by each agency or organization following the ICS guidelines of construction as a part of or a complement to an organization's emergency plan. An organization's emergency plan is comprised of various standard operating procedures (SOPs), associated checklists, reports, and actions to be taken in response to various emergencies. The ICS implementation is the organizational command and control layer that enables SOPs to be directed, executed and monitored. In practice, an organization's ICS structure is adopted in a pre-planned form (e.g., a pre-planned incident ICS). In some instances, there is no pre-planned ICS.

In many instances when an ICS is adopted and used use in an emergency, the instantiated ICS structure is commonly handwritten using grease boards, whiteboards, chart paper and other manual means to represent and visualize the implemented command structure. In some cases a computer software charting tool outputs to visually represent the ICS structure. To assess and make decisions, and to implement command directives and execute functions, communications and information coordination from the field to command, and from command to the field, and laterally among peer ICS nodes units are essential. As the ICS structure expands, the ability to efficiently establish formal communications and information sharing groups becomes increasingly difficult and time consuming, and in many cases are not well defined. The problem with establishing properly defined communication and information sharing groups in response to emergencies is well known and documented in the field and remains a significant unsolved problem. Further the current approaches are insufficient because an instantiated ICS is impermanent and can change during an incident. Assignments of personnel to node elements may change. In addition, certain functional domains represented by one or more ICS nodes may change status from being inactive, active or deactivated over the course of responding to and recovering from an emergency, based upon changing circumstances, conditions and other factors. The handwritten charts and even computer software charting tools do not solve the significant unsolved problem.

Some embodiments include a system, method, and computer programing product that automatically establishes defined communications and information sharing groups that become enabled or activated in response to an emergency or event. This accomplished by correlating members assigned to node elements within an adopted or instantiated isomorphic structure (e.g., ICS structure) as the basis for group identity and membership, and further enabling other defined communications groups which are either supersets or subsets of ICS nodes. By doing so, personnel assigned membership to and a position within an ICS node are automatically grouped as a defined communications group.

FIG. 1B illustrates example system 100B for hypergraphic self-defining communications groups of an isomorphic structure during an incident, in accordance with some embodiments of the disclosure. As a convenience and not a limitation, system 100B may be described with elements of other figures within the disclosure. System 100B includes IWS 110 of Hospital 1 and IWS 180 of Hospital 2. As described in FIG. 1A, when an incident occurs, IWS 110 can send invitation message 165 to IWS 180. After IWS 180 responds with an acceptance message, a communications group (e.g., a SIP session) is established between Hospital 1 and Hospital 2. IWS 110 determines which communications resources of Hospital 1 can join the communications group and IWS 180 determines which communications resources of Hospital 2 can join the communications group. The communications take place via connection 175, Interop Network 170, and connection 177.

IWS 110 of Hospital 1 can include a graphical user interface (GUI) that includes a member directory 120 that includes information regarding groups of personnel from Hospital 1 130, Hospital 2 150, local police 155, and an isomorphic structure 160. In some embodiments, isomorphic structure 160 can be a node, and can include example 700 of FIG. 7. As an example, Hospital 1 130 includes hospital security 135 that can include names, contact information, etc., of personnel that provide hospital security functions. The hospital office of emergency management (OEM) 140 can include groups such as director 142, planning 144, and public information 146, each of which can include one or more names and contact information of corresponding personnel. IWS 180 of Hospital 2 can include their respective personnel in member directory 182. Hospital 1 and hospital 2 can publish their respective directories with agencies and/or organizations with whom they have a relationship (e.g., a trusted relationship among trusted parties.) Thus, membership directory 120 can include hospital 2 personnel information that is published by hospital 2 and represented by hospital 2 150. In addition, hospital 1 and hospital 2 can also publish and share the types of incidents for which they have defined isomorphic structures for emergencies.

Isomorphic structure 160 (e.g., an ICS structure) can be dynamically scalable and morphologically pliable. Within an agency or organization, such as a local public safety or hospital, isomorphic structure 160 can be created with the intent of facilitating a well-organized and structured emergency response. The same construct may be applied to a joint or unified command environment where two or more agencies or organizations work together under an ICS structure and coordinate with one another under an emergency incident driven context. For example, isomorphic structure 186 of hospital 2 can be of the same construct as isomorphic structure 160 of hospital 1 such that the hospitals can work together under an emergency incident.

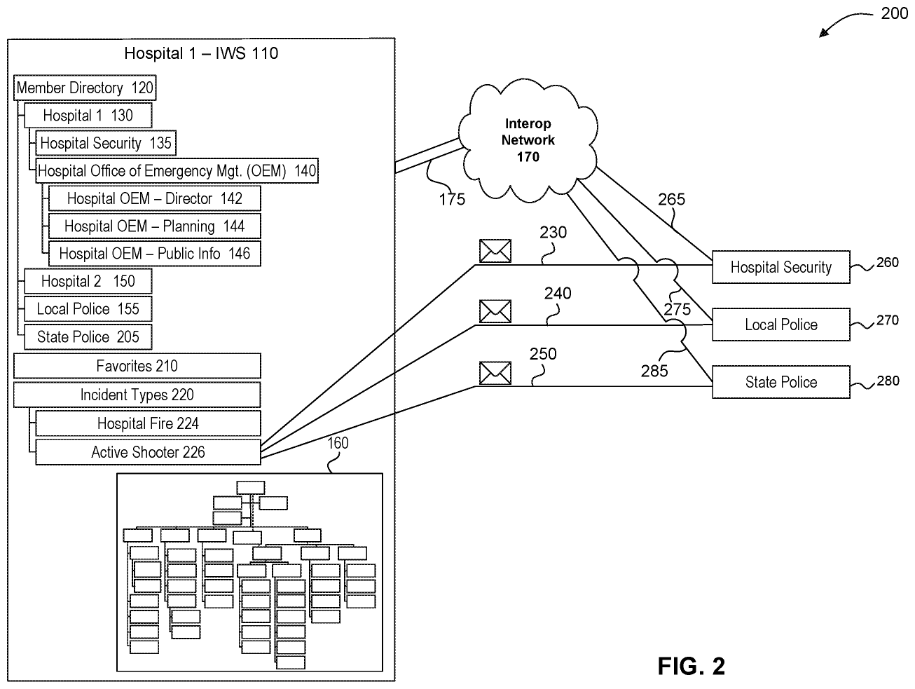

FIG. 2 illustrates example system 200 instantiating pre-defined communications groups during an incident, in accordance with some embodiments of the disclosure. As a convenience and not a limitation, system 200 may be described with elements of other figures in the disclosure. Membership directory 120 can also include other agencies like personnel (e.g., members) of state police 250, a list of favorites 210 of personnel that are contacted frequently, and incident types 220. Some incident types include hospital fire 224 and active shooter 226. A selection of active shooter 226 on a GUI of IWS 110 can instantiate a pre-defined communications group that includes transmitting invitation messages 230, 240, and 250 to hospital security 260, local police 270, and state police 280. After hospital security 260, local police 270, and state police 280 accept the respective invitations, a secure communications group can be established via connections 175, 265, 275, and 285, via interop network 170.

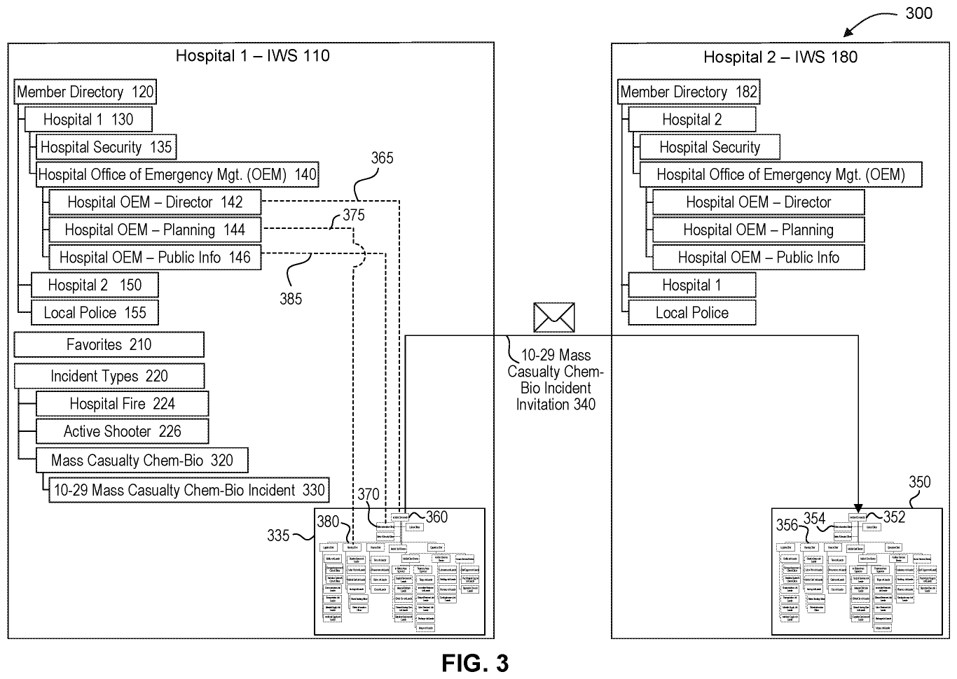

FIG. 3 illustrates example system 300 instantiating hypergraphic self-defining communications groups of an isomorphic structure during an incident, in accordance with some embodiments of the disclosure. As a convenience and not a limitation, system 300 may be described with elements from other figures in the disclosure. System 300 may include other types of incidents such as mass casualty chem-bio 320 that can be based on isomorphic structure 160. The type of specialists and specialized units needed for chemical and biological decontamination and treatment differ from an emergency requiring medical response to crush and burn injuries. When an incident occurs, a temporal instance of mass casualty chem-bio 320 can be created or instantiated shown as 10-29 mass casualty chem-bio incident 330. The instantiated instance, 10-29 mass casualty chem-bio incident 330, can be represented by an interactive visual chart (IVC) 335. In some embodiments IVC 335 represents a node and the rectangles within represent node elements. The rectangles can be selectable items of IVC 335. While shown as rectangles, the selectable items can be of a different shape and/or size.

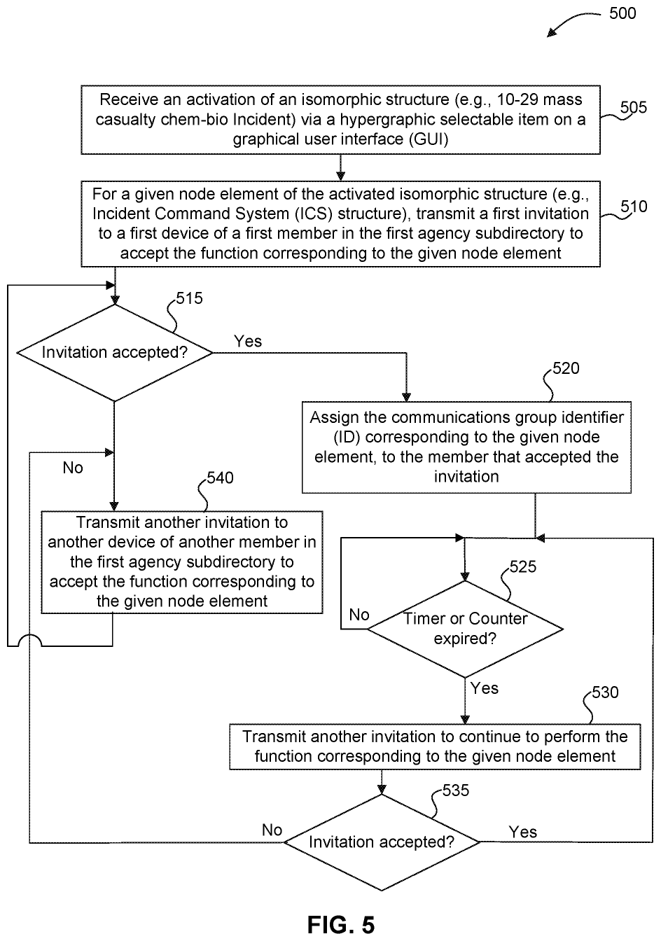

IVC 335 can be populated with personnel from member directory 120. For example, incident commander node element 360 (which can be incident commander 710 of FIG. 7) can be populated with personnel from the hospital OEM director 142 section of membership directory 120 as indicated by dotted line 365. Public information officer node element 370 (which can be public information officer 720 of FIG. 7) can be populated by personnel from the hospital OEM—public information 146 section as indicated by dotted line 385. And planning chief node element 380 (which can be planning chief 730 of FIG. 7) can be populated by personnel from the hospital OEM—planning 144 section as indicated by dotted line 375. More details regarding the populating are described in FIG. 5.

The instantiation of 10-29 mass casualty chem-bio incident 330 also includes creating two or more peer communications groups among agencies and/organizations that include a corresponding isomorphic structure to isomorphic structure 160. In example system 100B of FIG. 1B, hospital 2 includes isomorphic structure 186 which is a corresponding isomorphic structure to isomorphic structure 160. IWS 110 of hospital 1 can transmit invitation message 10-29 mass casualty chem-bio incident invitation 340 to IWS 180 of hospital 2. After IWS 180 accepts the invitation by transmitting an acceptance message (not shown), two or more peer communications groups can be instantiated where communications takes place via interop network 170 (not shown.) IWS 180 can create IVC 350 based on isomorphic structure 186 of system 100B, and populate corresponding functions of IVC 350 with appropriate hospital 2 personnel as reflected in member directory 182.

In some embodiments, when 10-29 mass casualty chem-bio incident 330 (e.g., a node) is instantiated, a communications group can be instantiated for each node element (e.g., each function represented by a selectable item such as a rectangle) of IVC 335. Two or more of the communications groups can be instantiated in parallel. The peer communications groups (e.g., peer talk groups or parallel communications sessions) that are instantiated can include the following: an incident commander communications group whose membership includes hospital 1 personnel assigned to incident commander node element 360, and hospital 2 personnel assigned to incident commander node element 352; a public information officer communications group whose membership includes hospital 1 personnel assigned to public information officer node element 370, and hospital 2 personnel assigned to public information officer node element 354; and a planning chief communications group whose membership includes hospital 1 personnel assigned to planning chief node element 380, and hospital 2 personnel assigned to planning chief node element 356. Subsequently, incident commander personnel from hospital 1 and hospital 2 can communicate via the incident commander communications group.

Further, IWS 110 can send invitation messages like 10-29 mass casualty chem-bio incident invitation 340 to other organizations (e.g., trusted parties not shown such as other hospitals, state police, and/or other agencies) that have a corresponding isomorphic structure 160. After the other organizations accept the invitation, the other organizations instantiate and populate corresponding IVCs. Their personnel corresponding to the respective functions (e.g., node elements such as incident commander node element 360) of IVC 335 can be included as members in corresponding communications groups that are instantiated by 10-29 mass casualty chem-bio incident 330. Accordingly, the corresponding personnel can join the incident commander communications group (e.g., talk group) and begin to communicate with incident commanders of node element 360 and node element 352, for example. Thus, the ability to instantiate multiple communications groups (e.g., multiple sessions in parallel) when an incident is instantiated (e.g., 10-29 mass casualty chem-bio incident 330) can save much time and effort, especially compared to the manual grease board or electronic bulletin board approaches.

In system 300, a local fire station and certain doctors of a local hospital may be marshalled to join the incident communications network as described in the Marshalling Patent. In the disclosure herein, however, the entirety of hospital 1 and the entirety of hospital 2 are defined within their respective activated incidents of isomorphic structures (e.g., ICS structures) and then their respective activated incidents of ICS structures (e.g., 10-29 mass casualty chem-bio incident 330 and IVC 335, and IVC 350) are aligned due to the isomorphic nature of the ICS structures. Thereafter, peer-to-peer communications groups across the respective activated incidents of ICS structures can be instantiated (e.g., two or more peer-to-peer communications groups are temporally instantiated substantially in parallel) to address the crisis. Further, membership within the peer-to-peer communications groups is fluid and determined by the respective agency (e.g., hospital 1 and hospital 2), not by a joint command structure commander (e.g., personnel change shifts, move to a different position, personnel are promoted, some resign, etc.) While isomorphic structure 160 can exist in a plan, the self-defining communications groups do not exist until 10-29 mass casualty chem-bio incident 330 is instantiated and individual agencies (e.g., hospitals 1 and 2) enable their respective isomorphic structure incidents (e.g., 10-29 mass casualty chem-bio incident 330, IVC 335 and IVC 350). Even within each agency's instantiated incident ICS structure (e.g., hospital 1's 10-29 mass casualty chem-bio incident 330, IVC 335), membership within corresponding self-defining communications groups can be fluid.

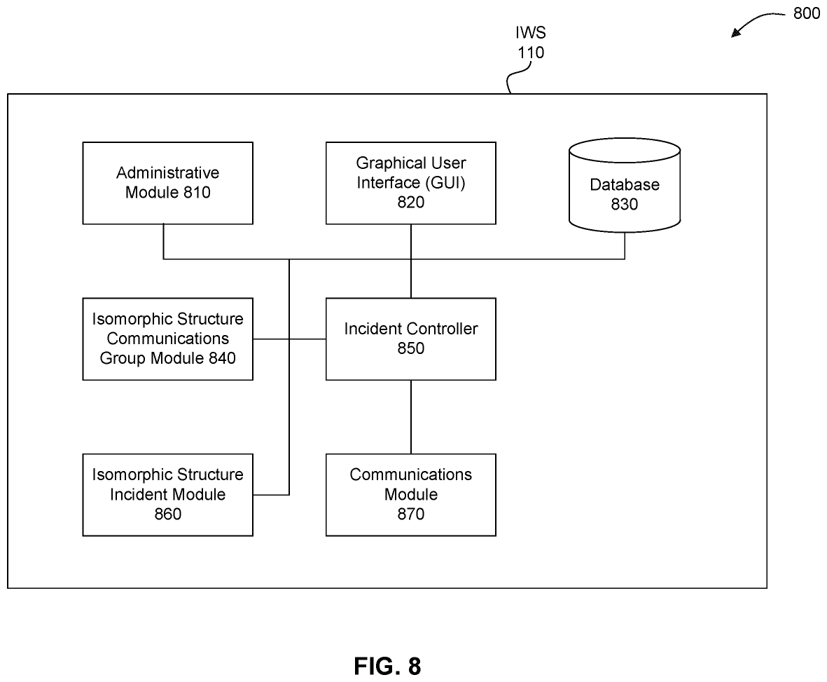

FIG. 8 illustrates an example block diagram of a system 800 for establishing hypergraphic self-defining communications groups of an isomorphic structure during an incident, in accordance with some embodiments of the disclosure. As a convenience and not a limitation, FIG. 8 can be described using elements of other figures in the disclosure. For example, system 800 can be IWS 110 or IWS 180 of FIG. 1B, or a mobile IWS (not shown.) System 800 can include incident controller 850 (e.g., one or more processors) that can execute instructions stored in memory (e.g., a computer readable medium) to perform the functions of the following: administrative module 810, graphical user interface (GUI) 820, isomorphic structure communications group module 840, isomorphic structure incident module 860, and communications module 870. Communications module 870 can include one or more network interfaces, including but not limited to wireless or wireline network interfaces and a variety of networking protocols.

Isomorphic structure incident module 860 can include a software application module coupled to database 830 that can be a data store, where an isomorphic structure (e.g., ICS structure 160) is graphically represented in a hierarchal or logically grouped manner. ICS structure 160 can be an object to which a unique identification and naming convention may be assigned, where the object can be comprised of at least one ICS node. Each ICS node may be related to another ICS node in a hierarchical relationship with one ICS node being the parent to one or more other child ICS nodes, or have no relational subordinated ICS nodes. Each ICS node may be assigned or associated with a functional domain or domains, geographic domain or other defining traits. An ICS node (e.g., ICS structure 160) may include of one or more node elements (e.g., node elements 360, 370, 380, 352, 354, 356) that can be objects having a unique ID and name. Each node element can include one or more positions which have a unique ID and associated name which may be an organizational title, job position or any other relevant designation to which a person may be assigned.

Administrative module 810 may include a function to assign personnel (e.g., hospital 1 personnel to various node elements of an instantiated isomorphic structure (e.g., 10-29 mass casualty chem-bio incident 330 represented by an interactive visual chart (IVC) 335.) For example, administrative module 810 may assign hospital OEM—director 142 to incident commander node element 360 as indicated by dotted line 365. Note that different types of incidents such as a fire may result in different personnel being assigned to a node element of incident commander. Additional assignments can occur but are not shown to simplify system 300. Each node element (e.g., incident commander node element 360, public information officer node element 370) may have associated properties including: a function and functional description; conditional qualifications of assignment such as organizational membership, employment or hire status, good standing, professional certification, skills, security clearance level, person availability, proximity, age, health, language skills, and/or any other qualifying factors relevant to the performance of the function assigned to the node element; time slots or periods to which persons may be assigned (e.g., particular shifts, temporary assignments); and/or identity information (e.g., node assignment identification (ID)) for any person assigned to a node element assignment.

The node assignment ID for any person assigned to a node element assignment can include the following information about the assignee: name; employer or member affiliation with an organization (e.g., hospital 1, local police); employer or member affiliation identification such as employee ID or badge number, or certification license number, incident-based and/or event personnel ID number or credential; personal identification information, including social security number, driver license number, passport number and/or other personally identifying information; personal health related data including disease, disease or exposure susceptibilities, and other health factors; and/or communications addressing and contact information of the assignee or proposed to be assignee to a node element.

Examples of the communications addressing and contact information of the assignee include but are not limited to: a member communications ID; telephone numbers and telephone session access codes; land mobile radio or push to talk network user or device IDs, channel ID, talk groups, call signs and/or other identifiers; IP network user ID, network addresses, including Universal Resource Locators, SIP Addresses and MAC, proxy addresses, and other relevant network connection protocol or required addressing information including Port IDs, encryption type, and/or tokens; email addresses; text and SMS user IDs; and/or social media user IDs.

For a node element assignment, a person or persons may be assigned to each node element (e.g., as a member) by an administrative user via the administrative module's graphical user interface (GUI) 820. For example, GUI 820 can display IVC 335 where the node elements are shown as selectable items—rectangles in system 300. The node element assignment may be a module of administrative module 810, where node assignment ID information of a member may be entered by an agent and stored. The node assignment ID module may be electronically linked to and retrieved to call or validate an assignee's node assignment ID Information from a separate or existing database 830, registry, directory or service which contains some or all of such information.

The node element assignment may include one or more rules or procedural steps including but not limited to the following: i) an agent may assign a member without prior permission or consent and send a notification to the member; ii) an agent may assign the member subject to the member (or another person with authority) granting consent or accepting the assignment; iii) an agent sending a request to a proposed member (or another person with authority) seeking consent or accepting the assignment; iv) an agent sending an invitation to one or more prospective assignees inviting a response to accept or reject an assignment. See FIG. 5. Sending can include the transmission of a message in any form, or is reducible to human readable or interpretable form, including oral, written, audible or visual means, by any medium, including electronic transmission, posting a message, or inviting a recipient inquiry with the intent to send a message or instruction.

Node element assignment may have a status such as pending, standby, active, suspended, disqualified, unconfirmed, confirmed, temporary or any other descriptive state of being. A node element assignment may be: pre-determined and assigned to a node element or initiated upon the activation of an ICS node (e.g., 10-29 mass casualty chem-bio incident 330) or the node element. The status a node element assignment may change based upon any triggering event, including a status change initiated by an agent, or may be automatically initiated by a status change in an ICS node or ICS object.

For any ICS object, isomorphic structure incident module 860 can include a function to assign or associate functional or operational emergency purpose or event context, which may include the nature or type of emergency or event, the place or severity of emergency or event, the geographic scope or size of the emergency or event, the declared status of the emergency event, or a role or purpose assigned or delegated to the ICS object by another entity for an emergency or event. For example, if a truck carrying toxic chemicals has an accident on a highway on October 29^{th}, and many people are affected, isomorphic structure incident module 860 can assign an instance of mass casualty chem-bio 320, 10-29 mass casualty chem-bio incident 330, to the toxic chemical truck highway accident.

For example, isomorphic structure incident module 860 can include a function to assign or associate, for any ICS object (e.g., 10-29 mass casualty chem-bio incident 330), a state of instantiation or purpose, including but not limited to the following: Being for operational use; being for training use; being for administrative use; being a template from which another distinct ICS object may be created by copying the object and corresponding ICS nodes and node elements and associated proprieties that may be further edited and modified; being a draft ICS object in process and not yet designated as completed; being approved or adopted by one or more authorities; being in a state of conformity with prevailing laws, rules, policies, practices, conventions or guidance or changes thereto; being out of date as a function of time; being subject to review as a function of time or review or other procedure; being suspended pending the fulfillment of prescribed conditions; being retired; and/or being renewed.

For any ICS node, node element or any grouping of ICS nodes or node elements comprising or associated with an ICS object, isomorphic structure incident module 860 can include a function to assign an operational state such as active, inactive, suspended, standby, deactivated, reactivated, or other designations describing the functional state of the ICS nodes (e.g., 10-29 mass casualty chem-bio incident 330) or node elements (e.g., incident commander node element 360, public information officer node element 370, or planning chief node element 380.) The functions of isomorphic structure incident module 860 can be performed via GUI 820.

Isomorphic structure communications group module 840 can include functions to create from an ICS node (e.g., 10-29 mass casualty chem-bio incident communications group 330), two or more distinct communications groups in parallel via an automated agent and/or a human agent. For example, isomorphic structure communications group module 840 can associate node element assignments to node elements within an ICS node, and assign a unique communications group identification (e.g., communications group ID) to the ICS node (e.g., 10-29 mass casualty chem-bio incident 330 communications group ID 001.)

In some embodiments isomorphic structure communications group module 840 registers the communications group ID with a group communications network and obtains, requests, or establishes and assigns individual user communication IDs for each member (e.g., a communications group user ID), where the group communications network may be a push to talk radio network, push to talk over mobile network, a voice or video conferencing call network, and/or an interoperable communications network (e.g., interop network 170) where more than one network communications medium may be interconnected or bridge with one or more others, such as but not limited to, a land mobile radio talk groups connected to or bridged to users of push to talk mobile clients.

In some embodiments isomorphic structure communications group module 840 uses the existing member communications IDs and associates them to bridge existing user communications addresses and modalities via an interoperable gateway, switching mechanism, communications server, or network that exchanges messages and communications among users from and to disparate network devices. For example, isomorphic structure communications group module 840 may create, register, and assign unique proxy communications group user IDs for each member that is relationally associated with existing member communications IDs.

Isomorphic structure communications group module 840 can include functions enabling visual display of a communications group ID and communications group user IDs associated with any GUI representation of an ICS object or ICS node (e.g., IVC 335.) Isomorphic structure communications group module 840 can initiate the creation of and/or create the communications group ID and communications group user IDs in any phase of instantiation of the ICS object or ICS node (e.g., 10-29 mass casualty chem-bio incident communications group 330) via a human user agent or automated agent. For example, by using rules based upon an occurrence of an event or change in state or status of event, where an occurrence or change in state or status of an event may be determined by any information communicated to, or observed by the agent or a proxy for the agent. The information communicated can include a message or notification sent from a third party or system to the agent signifying an occurrence of an event or change, including messages from a mass notification system, a private notification system, a warning system, an alarm system, an analytics system, a sensor system or a monitoring system.

Isomorphic structure communications group module 840 can include functions for sending a notification to a member informing the member of the member communications ID assigned to such person, and which may include a URL address, passcode, authentication code, a token, or key to include the following: i) Authenticate the member's identity or the member's communications device; ii) assign or receive an encryption key to any communications session associated to the communications group ID; iii) access a communications session associated to a communications group ID, send to other members, and/or receive from other members communications and associated information including but is not limited to: audio data, video data, and data content, transmitted in the communications session or associated with the communications session; iv) initiate a communications session associated to a communications group ID, and to invite other members to join the communications session; and/or be invited to a communications session associated to a communications group ID.

Isomorphic structure communications group module 840 can include functions to send a notification to a member informing the member of the status of a communications group associated with the communications group ID (e.g., status of the 10-29 mass casualty chem-bio incident communications group 330 communications group ID), which may include but is not limited to: Operating status such as active, activated, inactive, inactivated to indicate whether communications sessions may be initiated, are initiated or may no longer be initiated; and/or a member's user communications endpoint network sign-in status, network availability or presence, user's availability or readiness, and/or changes thereto.

Isomorphic structure communications group module 840 can include functions for a communications endpoint device with a user interface display, such as a mobile push to talk client or software dispatch console, to remotely register to the communications endpoint device and update the user interface to display, access, or call the corresponding ICS communications group or its member. After registering the communications endpoint device, the following updates including but not limited to the following may occur: associated communications application; local device or coupled network contact list; directory or address book; and/or display. Isomorphic structure communications group module 840 can update, change, or save a GUI representation of the group and members on the endpoint device GUI, including an icon, image, programmable object, URI, web link, UI control, plug-in, widget or text representation displaying the name or identity of the corresponding ICS communications group, and/or displaying the members of the ICS communications group. Such changes may be initiated by a function of isomorphic structure communications group module 840 sending a command to the communications end-point application server or switch. In some examples the changes can be sent from or retrieved from a file server or via a file service to be downloaded by the client device.

In some embodiments, isomorphic structure communications group module 840 can relationally associate an ICS communications group ID with an ICS object ID or ICS node IDs. An ICS communications group may be automatically assigned a name which is derived from the name assigned to an ICS object or ICS node, associated incident type or event or combination thereof.

Isomorphic structure communications group module 840 can include functions to represent and display an ICS communications group (e.g., an incident commander communications group including incident commander node element 360 and incident commander node element 352) as an application object, application control, or web link in the GUI representation of the ICS object (e.g., IVC 335), ICS node (e.g., IVC 335), or their subparts (e.g., selectable icons for node elements 360, 370, 380). The representation and display of an ICS communications group enables the following functions: Change of the status of an ICS communications group; initiate an ICS communications group session (e.g., two or more ICS communications group sessions in parallel); and/or participate in an ICS communications group session as a communications endpoint of a member where a communication module (e.g., communications module 870) is coupled to isomorphic structure communications group module 840 and GUI application (e.g., GUI 820). In some embodiments communications session controls are made part of the application (e.g., the application displaying the ICS communications group.) In some examples, external controls or objects, controls or application interface modalities are functionally, logically or visually associated with the application (e.g., the application displaying the ICS communications group). Examples of GUI 820 functions include but are not limited to: displaying, nesting, overlaying, or positional aligning of independent application windows, synchronizing, coordinating or ordering views to the forefront or background, minimizing and maximizing views, relative to others in a user's computer graphics display or through multiple screen displays.

Isomorphic structure communications group module 840 can include functions to enable an ICS communications group that can include one or more ICS nodes, ICS node elements, and/or members. The ICS communications group can be formed in any logical combination, including but not limited to: ICS node position or functional position equivalent groupings across one or more ICS nodes; exclusion of child or parent ICS nodes, or certain members of child or parent ICS nodes; inclusion of child or parent ICS nodes, or certain members of child or parent ICS nodes; grouping node elements or members by common or shared or continuity of time periods, shifts, functions, and/or duties.

In some embodiments, isomorphic structure incident module 860 can associate two or more ICS objects to form a new ICS object: The two or more ICS objects are logically joined with one being designated a parent; or the two or more ICS objects are each designated as node elements or members of third ICS object. In some examples, two or more ICS nodes associated with independent ICS objects may be associated and form a new ICS node: The two or more ICS nodes include one being designated a parent; or each ICS node being designated node elements or members of third ICS Node. In some embodiments, ICS communications groups may include a new parent-child ICS object relation or new parent-child ICS node relation; and/or a new third ICS Object or new third ICS Node.

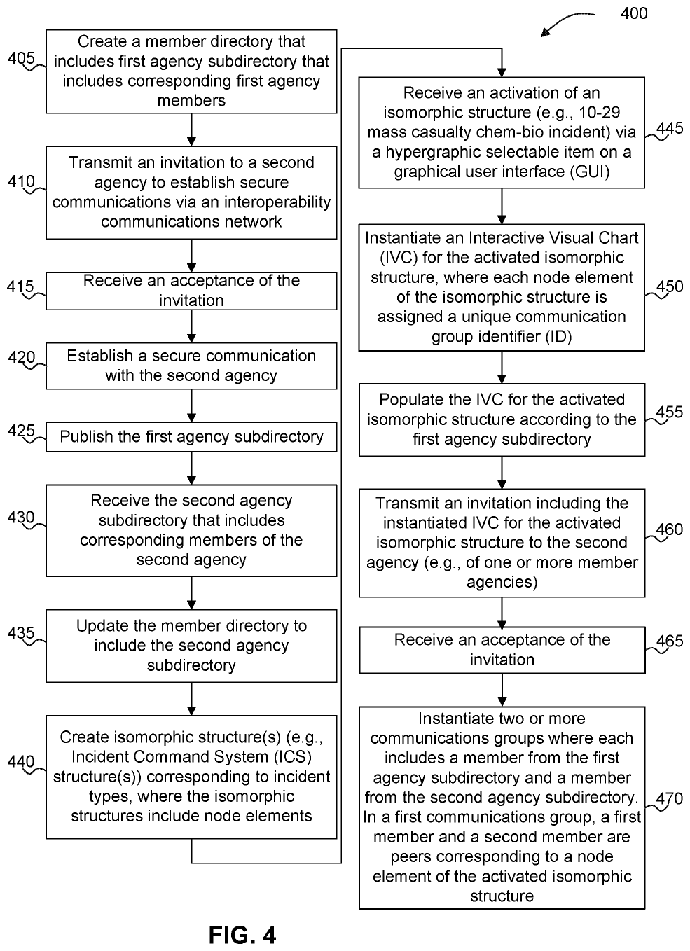

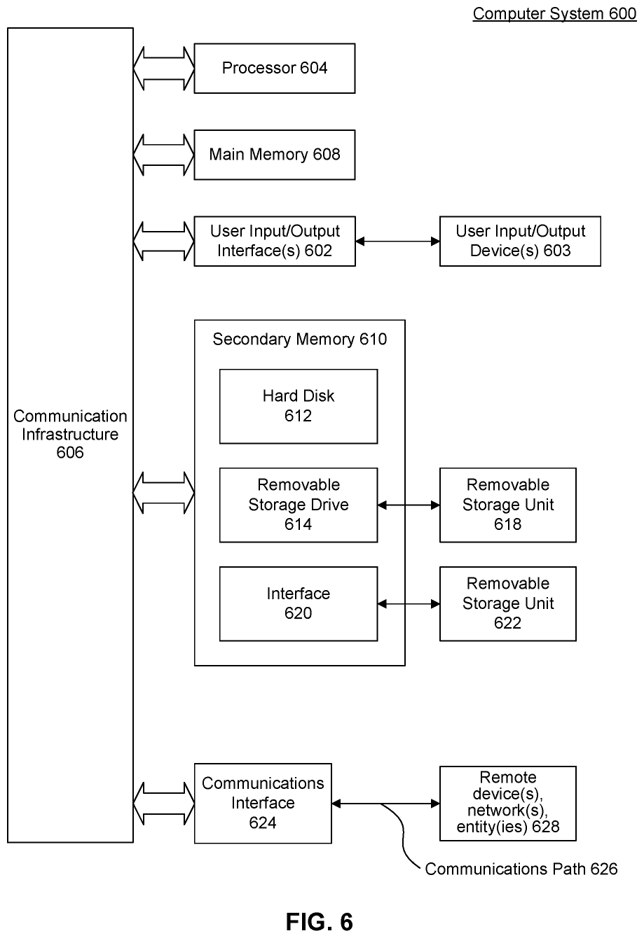

FIG. 4 illustrates method 400 for instantiating hypergraphic self-defining communications groups of an isomorphic structure during an incident, in accordance with some embodiments of the disclosure. As a convenience and not a limitation, method 400 may be described using elements from other figures in the disclosure. For example, method 400 may be performed by system 800 of FIG. 8 or example computer system 600 of FIG. 6.

At 405, system 800 creates a members directory that includes first agency subdirectory that includes corresponding first agency members. In system 100B, IWS 110 of hospital 1 creates member directory 120.

At 410, system 800 transmits an invitation to a second agency to establish secure communications via an interoperability communications network. In system 100B, IWS 110 of hospital 1 transmits incident invitation message 165 to IWS 180 of hospital 2 to establish secure communications via interop network 170.

At 415, system 800 receives an acceptance of the invitation. In system 100B, IWS 110 of hospital 1 receives an acceptance of the invitation from IWS 180 of hospital 2.

At 420, system 800 establishes a secure communication with the second agency. In system 100B, IWS 110 of hospital 1 establishes a secure communication with IWS 180 of hospital 2 via connection 175, interop network 170, and connection 177.

At 425, system 800 publishes the first agency subdirectory. In system 100B, IWS 110 publishes hospital 1 data of member directory 120. IWS 180 receives and includes the published hospital 1 data in member directory 182.

At 430, system 800 receives the second agency subdirectory that includes corresponding members of the second agency. In system 100B, IWS 180 publishes hospital 2 data of member directory 182.

At 435, system 800 updates the Member Directory to include the second agency subdirectory. In system 100B, IWS 110 receives and includes the published hospital 2 data in member directory 120 shown as hospital 2 150. Hospital 2 150 may include many subsections and branches that are not shown to simplify the illustration of system 100B.

At 440, system 800 creates isomorphic structure(s) (e.g., Incident Command System (ICS) structure(s)) corresponding to incident types, where the isomorphic structures include node elements. In system 100B, IWS 110 creates isomorphic structure 160 that includes node elements shown as rectangles. System 300 includes incident types 220 that includes different types of incidents including mass casualty chem-bio 320.

In some embodiments, system 800 publishes incident types 220. In system 100B (or system 300), IWS 180 receives the published incident types 220 and can determine that hospital 1 includes isomorphic structure 160 that corresponds to mass casualty chem-bio 320. In some embodiments hospital 2 creates isomorphic structure 186 that corresponds to isomorphic structure 160 of hospital 1. IWS 180 can also publish the incident types that hospital 2 supports (not shown). Accordingly, hospital 1 can be aware that hospital 2 includes isomorphic structure 186 that corresponds to isomorphic structure 160 that corresponds to mass casualty chem-bio 320. Thus, when 10-29-mass casualty chem-bio incident 330 is instantiated, IWS 110 of hospital 1 is aware of isomorphic structure 186, and transmits an invitation message to IWS 180 of hospital 2.

At 445, system 800 receives an activation of an isomorphic structure via a hypergraphic selectable item on a graphical user interface (GUI). In system 300, IWS 110 receives an activation of isomorphic structure 160. IWS 110 activates and instantiates an incident specific to the incident type, namely, 10-29 mass casualty chem-bio incident 330.

At 450, system 800 instantiates an Interactive Visual Chart (IVC) for the activated isomorphic structure, where each node element of the isomorphic structure is assigned a unique communications group identifier (ID). In system 300, IWS 110 instantiates IVC 335 corresponding to the activated 10-29 mass casualty chem-bio incident 330, which is assigned a unique communications group ID. In addition, node elements such as 360, 370, and 380 can each be assigned a unique communications group ID that also corresponds to the activated 10-29 mass casualty chem-bio incident 330 (e.g., 10-29 mass casualty chem-bio incident—incident commander node element 001.)

At 455, system 800 populates the IVC for the activated isomorphic structure according to the first agency subdirectory. In system 300, IWS 110 populates IVC 335 with hospital 1 personnel accordingly. Examples are shown by the dotted lines 365, 375, and 385.

At 460, system 800 transmits an invitation including the instantiated IVC for the activated isomorphic structure to the second agency (e.g., of one or more member agencies). In system 300, IWS 110 transmits 10-29 mass casualty chem-bio incident invitation 340 to IWS 180 that includes the unique communications group IDs for 10-29 mass casualty chem-bio incident 330 and node elements within (e.g., node elements 360, 370, and 380.)

At 465, system 800 receives an acceptance of the invitation. In system 300, IWS 110 receives an acceptance message from IWS 180. IWS 180 can instantiate isomorphic structure 186 of example 100B, and may generate IVC 350. IWS 180 can also populate IVR 350 with hospital 2 personnel according to member directory 182.

At 470, system 800 instantiates two or more communications groups where each communications group includes a member from the first agency subdirectory and a member from the second agency subdirectory. In a first communications group of the two or more communications groups, a first member of the first agency and a second member of the second agency are peers corresponding to a node element of the activated isomorphic structure. In system 300, IWS 110 instantiates two or more communications groups substantially in parallel. A communications group can be formed corresponding to one of the selectable items of IVC 335. For example, at least three communications groups associated with 10-29 mass casualty chem-bio incident 330 unique communications group ID are formed. An incident commander communications group that includes hospital 1 personnel assigned to node element 360 and hospital 2 personnel assigned to node element 352; a public information officer communications group that includes hospital 1 personnel assigned to node element 370 and hospital 2 personnel assigned to node element 354; and planning chief communications group that includes members including hospital 1 personnel assigned to node element 380 and hospital 2 members assigned to node element 356.

In some embodiments where IWS 110 determines that another organization (e.g., hospital 3 or a fire department in an adjacent town) does not have a pre-planned isomorphic structure (e.g., ICS) corresponding to isomorphic structure 160, IWS 110 can transmit an invitation that includes isomorphic structure 160 and/or 10-29 mass casualty chem-bio incident 330. Thus, the other organization can create an equivalent isomorphic structure, and instantiate a corresponding incident, where personnel from the other organization can subsequently join the parallel communications groups corresponding to IVC 335 and IVC 350.

FIG. 5 illustrates method 500 for populating a hypergraphic IVC of an isomorphic structure during an incident, in accordance with some embodiments of the disclosure. As a convenience and not a limitation, method 500 may be described using elements of other figures in the disclosure. For example, method 500 may be performed by system 800 of FIG. 8 or example computer system 600 of FIG. 6, or IWS 110 of system 300, or a mobile IWS (not shown.) In addition to enabling the initial assignment of personnel to various node elements of an activated instantiation of an isomorphic structure, method 500 enables the adjustment and adaptations needed over time as personnel change with the end/start of a work shift to swap assignments from retiring personnel to new personnel assuming existing functions. In some embodiments, as the incident progresses (e.g., escalates—method 500 adds additional staff, or de-escalates reduces staff, or other incident state changes as described above with regard to system 800.)

At 505, system 800 receives an activation of an isomorphic structure (e.g., 10-29 mass casualty chem-bio incident) via a selection of a hypergraphic selectable item on a graphical user interface (GUI). In system 300, IWS 110 may receive a selection of mass casualty chem-bio 320 followed by specific details to generate a specific instance, 10-29 mass casualty chem-bio incident 330. IWS 110 can generate IVC 335 that corresponds to instantiated 10-29 mass casualty chem-bio incident 330.

At 510, for a given node element of the activated isomorphic structure (e.g., Incident Command System (ICS) structure), system 800 transmits a first invitation to a first device of a first member in the first agency subdirectory to accept the function corresponding to the given node element. In system 300, IWS 110 may transmit a first invitation to a first device of one or more persons corresponding to hospital OEM-director 142 inviting them to accept assignment to incident commander node element 360. In some embodiments, IWS 110 may transmit the first invitation to a first device corresponding to a selected one (e.g., Jane Doe) of the one or more persons corresponding to hospital OEM-director 142 to accept assignment to incident commander node element 360. The first invitation may include information as described above with regard to administrative module 810 and isomorphic structure as described above with regard to isomorphic structure incident module 860 of system 800.

At 515, system 800 determines whether the invitation is accepted. In system 300, if Jane Doe accepts the invitation, method 500 proceeds to 520. Otherwise, method 500 proceeds to 546.

At 520, system 800 assigns the communications group ID corresponding to the given node element, to the member that accepted the invitation. In system 300, IWS 110 receives an acceptance from Jane Doe, and assigns a unique communications group ID corresponding to 10-29 mass casualty chem-bio incident 330 as well as node element 360 (e.g., 10-29 mass casualty chem-bio incident 330—incident commander node element 001.)

At 525, system 800 determines whether a timer or counter has expired. For example, a shift change may occur. In some embodiments, the incident may change a state (e.g., escalated, de-escalated, inactive). If a timer or counter has expired, system 800 proceeds to 530. Otherwise, system 800 remains at 525.

At 530, system 800 transmits another invitation to the personnel to continue to perform the function corresponding to the given node element. In system 300, IWS 110 transmits another invitation to Jane Doe inquiring whether she will remain in the next shift as incident commander.

At 535, system 800 determines whether the other invitation is accepted. If the other invitation is accepted, method 500 returns to 525. Otherwise, method 500 proceeds to 540.

At 540, when the other invitation is not accepted (e.g., Jane Doe will not remain in the next shift as incident commander), system 800 transmits another invitation to another device of another member in the first agency subdirectory to accept the function corresponding to the given node element. In system 300, assuming that the incident has not become inactive, IWS 110 transmits another invitation to invite different personnel associated with hospital OEM-director 142 to accept assignment to incident commander node element 360. Method 500 returns to 515 to determine whether the invitation is accepted.

Various embodiments can be implemented, by software, firmware, hardware, or a combination thereof. FIG. 6 illustrates and example computer system 600 in which the systems and devices described within various embodiments can be implemented as computer-readable code and/or text-readable code. After reading this description, it will become apparent to a person skilled in the relevant art how to implement the embodiments using other systems and/or processing architectures. For example, IWS 110 or IWS 180 of systems 100B, 200, and 300 as well as system 800 of FIG. 8 may be implemented by computer system 600.

Computer system 600 includes one or more processors (also called central processing units, or CPUs), such as a processor 604. Processor 604 is connected to a communication infrastructure 606 that can be a bus. One or more processors 604 may each be a graphics processing unit (GPU). In an embodiment, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 600 also includes user input/output device(s) 603, such as monitors, keyboards, pointing devices, etc., that communicate with communication infrastructure 606 through user input/output interface(s) 602. Computer system 600 also includes a main or primary memory 608, such as random access memory (RAM). Main memory 608 may include one or more levels of cache. Main memory 608 has stored therein control logic (i.e., computer software) and/or data.

Computer system 600 may also include one or more secondary storage devices or memory 610. Secondary memory 610 may include, for example, a hard disk drive 612 and/or a removable storage device or drive 614. Removable storage drive 614 may be a floppy disk drive, a magnetic tape drive, a compact disk drive, an optical storage device, tape backup device, and/or any other storage device/drive.

Removable storage drive 614 may interact with a removable storage unit 618. Removable storage unit 618 includes a computer usable or readable storage device having stored thereon computer software (control logic) and/or data. Removable storage unit 618 may be a floppy disk, magnetic tape, compact disk, DVD, optical storage disk, and/ any other computer data storage device. Removable storage drive 614 reads from and/or writes to removable storage unit 618 in a well-known manner.

According to an exemplary embodiment, secondary memory 610 may include other means, instrumentalities or other approaches for allowing computer programs and/or other instructions and/or data to be accessed by computer system 600. Such means, instrumentalities or other approaches may include, for example, a removable storage unit 622 and an interface 620. Examples of the removable storage unit 622 and the interface 620 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 600 may further include a communication or network interface 624. Communication interface 624 enables computer system 600 to communicate and interact with any combination of remote devices, remote networks, remote entities, etc., (individually and collectively referenced by reference number 628). For example, communication interface 624 may allow computer system 600 to communicate with remote devices 628 over communications path 626, which may be wired and/or wireless, and which may include any combination of LANs, WANs, the Internet, etc. Control logic and/or data may be transmitted to and from computer system 600 via communication path 626.

In an embodiment, a tangible, non-transitory apparatus or article of manufacture comprising a tangible, non-transitory computer useable or readable medium having control logic (software) stored thereon is also referred to herein as a computer program product or program storage device. This includes, but is not limited to, computer system 600, main memory 608, secondary memory 610, and removable storage units 618 and 622, as well as tangible articles of manufacture embodying any combination of the foregoing. Such control logic, when executed by one or more data processing devices (such as computer system 600), causes such data processing devices to operate as described herein.

Based on the teachings contained in this disclosure, it will be apparent to persons skilled in the relevant art(s) how to make and use embodiments of the invention using data processing devices, computer systems and/or computer architectures. In particular, embodiments may operate with software, hardware, and/or operating system implementations other than those described herein.

### CONCLUSION

It is to be appreciated that the Detailed Description section, and not the Summary and Abstract sections (if any), is intended to be used to interpret the claims. The Summary and Abstract sections (if any) may set forth one or more but not all exemplary embodiments of the invention as contemplated by the inventor(s), and thus, are not intended to limit the invention or the appended claims in any way.

While the invention has been described herein with reference to exemplary embodiments for exemplary fields and applications, it should be understood that the invention is not limited thereto. Other embodiments and modifications thereto are possible, and are within the scope and spirit of the invention. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments may perform functional blocks, steps, operations, methods, etc., using orderings different than those described herein.

The breadth and scope of the invention should not be limited by any of the above-described exemplary embodiments, but should be defined only in accordance with the following claims and their equivalents.

What is claimed is:

1. A system for a first agency, comprising:
a transceiver; and
one or more processors coupled to the transceiver, configured to:
establish, via the transceiver, communications with a second agency via an interoperability communications network;
create an isomorphic structure comprising a first and a second node element in a hierarchy;
instantiate an interactive visual chart (IVC) for the isomorphic structure that is activated, wherein the first node element corresponds to a first communications group identifier (ID) and the second node element corresponds to a second communications group ID;
transmit, via the transceiver, a first invitation including the instantiated IVC for the activated isomorphic structure; and
in response to the first invitation being accepted, instantiate a first communications group corresponding to the first communications group ID and a second communications group corresponding to the second communications group ID, wherein the first communications group includes a first member of the first agency and a second member of the second agency, wherein the first member and the second member are peers corresponding to the first node element of the activated isomorphic structure.

2. The system of claim 1, wherein the activated isomorphic structure comprises an Incident Command System (ICS) structure.

3. The system of claim 1, wherein the one or more processors are further configured to:
populate the IVC for the activated isomorphic structure with members of the first agency.

4. The system of claim 3, wherein to populate the IVC for the activated isomorphic structure, the one or more processors are configured to:
transmit, via the transceiver, a second invitation to a first device of the first member of the first agency to accept a function corresponding to the first node element; and
in response to the second invitation being accepted, assign the first communications group ID to the first device or the first member.

5. The system of claim 4, wherein the one or more processors are further configured to:
transmit, via the transceiver, a third invitation to the first device of the first member to continue to accept the function corresponding to the first node element;
in response to the third invitation being denied, transmit a fourth invitation to an other device of an other member of the first agency to accept the function corresponding to the first node element; and
in response to the fourth invitation being accepted, reassign the first communications group ID to the other device or the other member.

6. The system of claim 1, further comprising:
a graphical user interface (GUI) coupled to the one or more processors, wherein the GUI comprises hypergraphic selectable items corresponding one or more created isomorphic structures including the isomorphic structure, wherein the one or more processors are configured to:
receive, via the GUI, a first selection of a first hypergraphic selectable item corresponding to the isomorphic structure, of the hypergraphic selectable items; and
activate the isomorphic structure.

7. The system of claim 6, wherein the one or more processors are further configured to:
receive, via the GUI, a second selection of a second hypergraphic selectable item corresponding to the first node element of the instantiated IVC for the activated isomorphic structure;
in response to the reception of the second selection of the second hypergraphic selectable item, transmit, via the transceiver, a second invitation to a first device of the first member of the first agency to accept a function corresponding to the first node element; and
in response to the second invitation being accepted, assign the first communications group ID to the first device or the first member.

8. The system of claim 1, wherein the first communications group ID corresponds to: push to talk radio network, push to talk over mobile network, a voice or video conferencing call network.

9. The system of claim 1, wherein the one or more processors are further configured to:
instantiate parallel sessions that include peer members from the first agency and a third agency coupled to the interoperability communications network, wherein the peer members correspond to respective node elements in the hierarchy of the activated isomorphic structure.

10. The system of claim 1, wherein the one or more processors are further configured to:
create a member directory comprising a first agency subdirectory;
publish the first agency subdirectory;
receive a second agency subdirectory; and
update the member directory to include the second agency subdirectory.

11. A method for a system of a first agency, comprising:
establishing communications with a second agency via an interoperability communications network;
creating an isomorphic structure comprising a first and a second node element in a hierarchy;
instantiating an interactive visual chart (IVC) for the isomorphic structure that is activated, wherein the first node element corresponds to a first communications group identifier (ID) and the second node element corresponds to a second communications group ID;
transmitting a first invitation including the instantiated IVC for the activated isomorphic structure; and
in response to the first invitation being accepted, instantiating a first communications group corresponding to the first communications group ID and a second communications group corresponding to the second communications group ID, wherein the first communications group includes a first member of the first agency and a second member of the second agency, wherein the first member and the second member are peers corresponding to the first node element of the activated isomorphic structure.

12. The method of claim 11, wherein the activated isomorphic structure comprises an Incident Command System (ICS) structure.

13. The method of claim 11, further comprising:
populating the IVC for the activated isomorphic structure with members of the first agency.

14. The method of claim 13, wherein the populating the IVC for the activated isomorphic structure comprises:
transmitting a second invitation to a first device of the first member of the first agency to accept a function corresponding to the first node element; and
in response to the second invitation being accepted, assigning the first communications group ID to the first device or the first member.

15. The method of claim 14, further comprising:
transmitting a third invitation to the first device of the first member to continue to accept the function corresponding to the first node element;
in response to the third invitation being denied, transmitting a fourth invitation to an other device of an other member of the first agency to accept the function corresponding to the first node element; and
in response to the fourth invitation being accepted, reassigning the first communications group ID to the other device or the other member.

16. The method of claim 11, further comprising:
displaying the instantiated IVC for the activated isomorphic structure via a graphical user interface (GUI) comprising hypergraphic selectable items corresponding to one or more isomorphic structures including the isomorphic structure;
receiving, via the GUI, a first selection of a first hypergraphic selectable item corresponding to the isomorphic structure, of the hypergraphic selectable items; and
activating the isomorphic structure.

17. The method of claim 16, further comprising:
receiving, via the GUI, a second selection of a second hypergraphic selectable item corresponding to the first node element of the instantiated IVC for the activated isomorphic structure;
in response to receiving the second selection of the hypergraphic selectable item, transmitting a second invitation to a first device of the first member of the first agency to accept a function corresponding to the first node element; and
in response to the second invitation being accepted, assigning the first communications group ID to the first device or the first member.

18. The method of claim 11, wherein the first communications group ID corresponds to: a push to talk radio network, push to talk over mobile network, a voice or video conferencing call network.

19. The method of claim 11, further comprising:
instantiating parallel sessions that include peer members from the first agency and a third agency coupled to the interoperability communications network, wherein the peer members correspond to respective node elements in the hierarchy of the activated isomorphic structure.

20. A non-transitory computer-readable medium storing instructions that, when executed by a processor of a first electronic device of a first agency, cause the processor to perform operations, the operations comprising:
establishing communications with a second agency via an interoperability communications network;
creating an isomorphic structure comprising a first and a second node element in a hierarchy;
instantiating an interactive visual chart (IVC) for the isomorphic structure that is activated, wherein the first node element corresponds to a first communications group identifier (ID) and the second node element corresponds to a second communications group ID;
transmitting a first invitation including the instantiated IVC for the activated isomorphic structure; and
in response to the first invitation being accepted, instantiating a first communications group corresponding to the first communications group ID and a second communications group corresponding to the second communications group ID, wherein the first communications group includes a first member of the first agency and a second member of the second agency, wherein the first member and the second member are peers corresponding to the first node element of the activated isomorphic structure.
